(19) United States
(12) Patent Application Publication
(10) Pub. No.: US 2011/0046637 A1
(43) Pub. Date: Feb. 24, 2011

(54) SENSORIZED MEDICAL INSTRUMENT

(75) Inventors: Rajnikant Patel, London (CA); Ana Luisa Trejos, London (CA); Mahdi Tavakoli, Edmonton (CA); Michael David Naish, London (CA)

Correspondence Address: BACON & THOMAS, PLLC, 625 SLATERS LANE, FOURTH FLOOR, ALEXANDRIA, VA, 22314-1176, US

(73) Assignee: THE UNIVERSITY OF WESTERN ONTARIO, London (GB)

(21) Appl. No.: 12/812,867

(22) PCT Filed: Jan. 8, 2009
(86) PCT No.: PCT/CA2009/000021

§ 371 Date: Nov. 8, 2010

Related U.S. Application Data
(60) Provisional application No. 61/006,443, filed on Jan. 14, 2008.

(51) Int. Cl. A61B 19/00

(52) U.S. Cl. 606/130

(57) ABSTRACT

The present invention provides a sensorized medical instrument that is suitable for application to, and training and skills assessment for, a variety of therapeutic, diagnostic, surgical and medical procedures. It is comprised of a sterilizable sensorized instrument capable of measuring forces in five degrees of freedom and tip position in six degrees of freedom. The instrument can be used to perform a variety of tasks through the integration of interchangeable instrument tips and handles. The system is capable of providing feedback to the user regarding critical forces/torques acting on the tissue, position and orientation of the instrument and its tip near critical areas within the body, or user performance during training and skills evaluation.

### CROSS REFERENCE TO RELATED U.S. APPLICATIONS

This patent application relates to, and claims the priority benefit from, U.S. Provisional Patent Application Ser. No. 61/006,443 filed on Jan. 14, 2008, in English, entitled TRAINING AND SKILLS ASSESSMENT SYSTEM FOR MINIMALLY INVASIVE SURGERY, and which is incorporated herein by reference in its entirety.

### FIELD OF THE INVENTION

The present invention relates to sensorized medical instruments that are suitable for application to, and training and skills assessment for, a variety of therapeutic, diagnostic, surgical and medical procedures.

### BACKGROUND OF THE INVENTION

A type of Minimally Invasive Surgery (MIS), called endoscopic surgery, refers to the use of long slender instruments and a camera that enter the patient's body through small incisions (1 cm in diameter). This type of surgery is also called laparoscopic surgery when performed in the abdomen, or thoracoscopic surgery when performed in the thorax. Compared to open surgery, MIS significantly reduces tissue trauma, post-operative pain and recovery time. Unfortunately, the “fulcrum effect” generated at the entry site causes a significant reduction in dexterity and reversal of hand motion, requires higher manipulation forces to overcome the drag on the instruments, and considerably degrades haptic feedback (the sense of touch). These limitations result in new perceptual-motor relationships which reduce performance and are unfamiliar to the user.

The minimally invasive access in diagnostic, therapeutic and surgical procedures results in limitations that affect the ability of the user to sense forces applied by the instrument. In addition, the limited field of view of the operating site (such as that produced through use of an endoscopic camera) may cause the user to become disoriented within the surgical environment. The ability to detect forces and position in surgical procedures, combined with suitable feedback to the user, could mitigate the challenges of MIS techniques.

Furthermore, the widespread application of MIS is hindered by the lack of appropriate educational and training tools. During conventional surgical training, a trainee first watches a skilled professional doing an operation and then tries the operation under the guidance of the mentor. This mode of training is inefficient and the required perceptual-motor skills therefore take longer than normal to master and pose a challenge to surgeons interested in acquiring these skills. A possible solution is to develop training exercises that can be used to develop the skills necessary, while providing immediate feedback to the user on their achieved performance.

The ability to sense forces and torques, as well as position, during MIS would allow the development of systems that solve these problems by providing real-time feedback during medical procedures or during training sessions. A review of what has been done in the area is presented below.

### Force Sensing

Force sensing systems for minimally invasive surgery have stringent design specifications that have limited their development. The specifications include the following [1,2]: the instruments must be less than 10 mm in diameter, which considerably restricts the size of the sensors; high friction at the trocar (entry point) makes it hard to sense from outside of the body; the fact that different instruments are used in each procedure means that the instruments must be versatile; actuation of the gripper requires the sensor to be hollow; and sterilizability means that the sensor should not be applied directly to the jaws.

A few researchers have approached the force sensing issue by incorporating sensors directly onto the gripper. A laparoscopic tactile sensor with a piezoelectric polymer PVDF film is proposed in [3]. The tactile sensor has 0.1 N sensitivity. Reference [4] proposes a tactile sensor attached to the tip of an endoscope that determines forces by measuring displacements between a transparent window and the end of the endoscope using image processing. A two dimensional mechanical sensor to measure thrust and pull inside instrument jaws is proposed in [5]. The design of a laparoscopic grasper is proposed in [6], which uses piezoelectric sensors to detect forces in three degrees of freedom; however, the instrument is quite large for this application (the size of a quarter). [7] used finite element analysis to evaluate the performance of a tooth-like sensor. Miniaturization of this device is still required.

Some researchers have tried sensing the forces on the handle of the instrument. The use of a mechanical arm to hold the instrument is proposed in [1] in which an overcoat device eliminates forces caused by the trocar. An innovative measuring instrument intended for the quantitative analysis of the laparoscopic operations actually performed is proposed in [8]. A laparoscopic grasper (95 g) was modified with a 6-axis position sensor (PC Bird) and a mini force torque sensor (ATI Industrial Automation, total weight 261 g). Although this system has been designed for training purposes, forces are measures outside of the trocar. A sensorized instrument used for haptic recording is proposed in [9]. It serves to record tool tissue interactions from actual procedures for later use in haptic simulators. In [10], a sensorized grasper was developed with a 6-DOF mini sensor (ATI Industrial Automation), plus another force sensor (a FUTEK FR1010) on the grasper handle. Finally, in [11], actual minimally invasive surgical tools were modified by adding two strain gauges onto a sensing module and are used to estimate the properties of the manipulated tissue. Position sensing is accomplished using an optical sensing device.

Other researchers have tried to sense forces directly on the shaft of the instrument. For example, [12] proposes a high-accuracy novel 3 DOF miniature force sensor 12.5 mm in diameter×15 mm long for internal sensing of tip forces.

Some researchers have tried to incorporate force sensing into a master-slave setup [1,13,14]. The development of a master/slave system capable of providing haptic feedback is presented in [15].

The integration of force and position sensing for training purposes was incorporated into the BlueDRAGON [16]. Two four bar linkages equipped with position and force sensors acquire the kinematics and dynamics of two endoscopic tools. Position in four of the mechanism joints is measured via potentiometers, forces and torque are measured through an ATI mini sensor, and tool tissue interaction is measured via contact sensors. This system measures manipulation forces, not tool tissue forces. Furthermore, there is no consideration of the effect that the manipulator would have on the normal movement of the laparoscopic instruments.

In [17], Markov modeling is used together with the BlueDRAGON to model the complexity of MIS for surgical skill analysis to obtain an objective quantification of skill defined as the statistical similarity of a data measured from a subject with apparently unknown skill level to an expert and a novice surgeon. A surgical skill evaluation system based on this work has been patented [18] and is commercially available as the EDGE system (Simulab Corp., Seattle, Wash.).

### Position Sensing

Research involving the use of electromagnetic, ultrasound and fluoroscopic tracking has been reported in the literature [19-25]. Unfortunately, these systems are reported to be cumbersome and expensive. There has also been significant headway made into the realm of optical tracking, which is readily commercially available. In these studies, the tracking systems were never utilized during training, and acted only as an aid to surgeons in the operating room [22]. The accuracy of electromagnetic trackers has been known to change over time, which could result in a critical error during surgery [23, 24]. Closely related to magnetic tracking is sonic-based tracking, which can be very accurate in detecting instrument movement. The problem with this method is that it is very sensitive to movement, leading to inaccurate results. Additionally, the instruments could be out of the “range of hearing” of the sensor, so careful sensor placement is integral to the functionally of the system [26].

Ideally, a solution to the problem of surgical instrument tracking in three-dimensional space would use only the images acquired by the endoscope. Unfortunately, the current techniques deployed to track surgical instruments using digital image processing are insufficient for medical users, as they do not provide any depth perception from the endoscopic image.

The bulk of current research into real time surgical instrument tracking using digital image processing without the use of large sensor arrays or bulky equipment utilizes markers (usually in the form of LED's) placed on the instrument, to determine the position of the instrument in space.

Position tracking for surgical training purposes, deals mostly with the development of surgical simulators. These, however, do not incorporate three dimensional aspects, making minimally invasive surgery difficult to learn. There has also been some research into virtual reality simulations for surgical training using tracking. A team processed endoscopic images into three dimensions to create surgical simulations [27]. The major drawback to this technique is that it is primarily for texture mapping in virtual reality simulators and is not feasible for real time instrument tracking.

### Active Marker Based Tracking

One group [28] used two blinking LED's embedded on the instrument, which alternated turning on and off at a frequency identical to the endoscope. An algorithm was then implemented to detect light changes in the endoscopic images, and an expression for the two-dimensional orientation of the instrument was derived. The reason active markers were used was that lighting inside a human cavity is unpredictable, due to varying textures of organs and uneven background lighting. The problem with this method is that it only offers a two dimensional orientation of the instrument.

Another team [29] tried to solve the three-dimensional problem. LEDs were mounted on the surgical instrument and set to blink at the endoscope frequency; lasers were mounted on the tool and a projection was produced onto the tissue surface. Four points were projected onto the organ surface to minimize error, and from this data the position of the surgical instrument was found using a similar algorithm to the one presented in [28]. The additional information that the laser projection provided was enough to derive a formula for the estimated depth of the surgical instrument. The drawback of this approach is that the markers used on the system are active, the algorithm only works because the LEDs or lasers blink. The reason that passive markers are desirable over active ones is that active markers add a power requirement to the surgical tools, and any added weight/wiring to the instrument is undesirable.

### Visual Servoing

One team [30] utilized color image segmentation in their design to detect the surgical instrument from a digital image. They present a study of laparoscopic images and videos, and compiled color information. Other groups [26, 30, 31] tracked the shape of the instrument moving through tissue. The images were processed by analyzing individual pixels and comparing them to create general shapes. These shapes were then further analyzed, and an algorithm was established to determine which shape was the instrument. The results from this image analysis were carried over to the next image, to speed up processing time. Results have shown that the initial processing time had a delay of approximately three seconds. Some drawbacks to this technique were that the team was not concerned about positioning of the instrument, and often the tool was only detected as a blurred image.

Most other servoing research has been utilizing optical tracking systems, known as “outside in” systems [21], meaning that they track the movement of the surgical device by plotting it relative to externally placed sensors whose location in space is already known. This solution is feasible for three-dimensional tracking within a training environment, but its practical uses for surgery are limited at best. There are several commercially available optical tracking systems which are “inside out” systems, meaning that the object being tracked is embedded with markers, and its position relative to other known sensors is measured. This solution is more fitting for a surgical setting, although the use of external hardware is undesirable.

### Passive Markers

Researchers have tried to use passive marking systems in combination with servoing algorithms to accurately track surgical tools [32]. The researchers used color segmentation of digital endoscopic images, and analyzed them in hue-saturation (H-S) instead of Red-Green-Blue (RGB) format. From this, a color was selected which did not appear on any of the H-S maps. This color (a light blue) was then used to mark the surgical instrument. The result of these experiments is very promising, although the precision of the algorithm is not ideal.

### SUMMARY OF THE INVENTION

The present invention provides a slender, sterilizable sensorized instrument capable of measuring tip forces in, but not limited to, five degrees of freedom and tip position in, but not limited to, six degrees of freedom.

The present invention is very advantageous in that it provides these sensing abilities, while maintaining the size, weight, balance, functionality, and sterilizability characteristics of conventional medical instruments. This configuration allows the instrument to be used in all of the environments where the use of conventional instruments have become standard, including, but not limited to, live animals and humans.

The instrument can be used to perform a variety of tasks through the integration of interchangeable instrument tips and handles. The system is capable of providing feedback to the operator regarding position of the instrument (e.g., its proximity to critical structures) and the forces being generated at the tool tip. This information can be used in many different ways to enhance the medical procedure, for example it could serve to warn the operator of potentially unsafe operating practice, or it could be used to provide real-time feedback on user performance. Furthermore, the data measured by the instrument may be recorded for subsequent evaluation or for use in training. The design of the instrument enables quantitative feedback to be provided during real procedures (e.g., surgery, therapy or diagnosis in animals or humans), offering a significant benefit over other training systems that must rely on simulated (physical or virtual) environments. The ability to record force and position profiles of experts performing real procedures allows the development of training environments that employ the use of virtual fixtures in which support is provided to the user in varying degrees, depending on their demonstrated abilities.

Thus the present invention provides a sensorized instrument system, comprising:

a) an instrument with an interchangeable handle, a shaft assembly operably connected at a proximal end portion to the interchangeable handle and attached at a distal end portion to an interchangeable tool, said interchangeable handle, shaft assembly and interchangeable tool operably connected so that the interchangeable handle controls actions of the interchangeable tool, said shaft assembly being configured to rotate the tool with respect to the interchangeable handle;

b) said instrument capable of accommodating tools for surgical, diagnostic, therapeutic and general medical procedures;

c) force/torque sensing means incorporated into the shaft assembly and positioned to provide measurement of kinesthetic or tactile forces/torques in, but not limited to, 5 degrees of freedom (DOFs), said force/torque sensing means being positioned and configured to measure kinesthetic and/or tactile forces/torques acting on the interchangeable tool that interacts with tissue;

d) first position sensing means for tracking position and orientation in 6 degrees of freedom (DOFs) of a tool tip of the interchangeable tool;

e) second position sensing means for tracking an open/close angle of the interchangeable tool tip;

f) a means of sealing the sensing elements so as to allow the instrument to be sterilized;

g) a computer system incorporating appropriate hardware, software, and algorithms, connected to said sensing means and said first and second position sensing means, said computer system configured to integrate acquired kinesthetic or tactile force/torque and position information when a user is performing medical procedures;

h) said computer system configured to record forces/torques applied to the instrument and a tool tip position for subsequent analysis and/or integration into other medical and/or training systems; and

i) said computer system configured to provide feedback to a user of the instrument regarding the instrument position and forces being exerted on tissue undergoing manipulation.

The present invention also provides a sensorized instrument system, comprising:

a) a hand-held minimally invasive instrument with an interchangeable handle, an inner shaft attached at a proximal end to the interchangeable handle and attached at a distal end to an interchangeable tool, said interchangeable handle, inner shaft and interchangeable minimally invasive tool operably connected so that the interchangeable handle controls actions of the interchangeable tool;

b) said minimally invasive instrument capable of accommodating tools for surgical, diagnostic, therapeutic and general medical procedures;

c) a middle shaft in which the inner shaft is movable for connecting the interchangeable handle and the interchangeable minimally invasive tool;

d) force/torque sensing means incorporated into the middle and the inner shafts and positioned to provide measurement of kinesthetic or tactile forces/torques in at least 5 degrees of freedom (DOFs), said plurality of force/torque sensors being positioned and configured to measure kinesthetic or tactile forces/torques acting on the interchangeable minimally invasive tool that interacts with tissue;

e) first position sensing means for tracking position and orientation in 6 degrees of freedom of a tool tip of the interchangeable minimally invasive tool and the intermediate joints of a redundant surgical shaft;

f) second position sensing means for tracking an open/close angle of the instrument tip;

g) a means of sealing the sensing elements so as to allow the minimally invasive instrument to be sterilized;

h) a computer system incorporating appropriate hardware, software, and algorithms, connected to said force/torque sensing means and said first and second position sensing means, said computer system configured to integrate acquired kinesthetic or tactile force/torque and position information when a user is performing medical procedures; and

i) said computer system capable of recording the forces/torques applied to the instrument and the tip position for subsequent analysis or integration into other medical and/or training systems; and

j) said computer system configured to provide feedback to a user of the instrument regarding the instrument position and forces/torques being exerted on tissue undergoing manipulation.

The present invention also provides a sensorized instrument system, comprising:

a) an instrument that articulates to provide additional degrees of degrees of freedom beyond the 5 degrees of freedom of motion available with a conventional minimally invasive instrument, with an interchangeable handle, a shaft assembly operably connected at the proximal end portion to the interchangeable handle and attached at a distal end portion to an interchangeable tool, said interchangeable handle, shaft assembly and interchangeable tool operably connected so that the interchangeable handle controls actions of the interchangeable tool, said shaft assembly being configured to rotate the interchangeable tool with respect to the interchangeable handle;

b) said instrument capable of accommodating tools for surgical, diagnostic, therapeutic and general medical procedures;

c) force/torque sensing means incorporated into the shaft assembly and positioned to provide measurement of kinesthetic or tactile forces/torques in any number of degrees of freedom, said force/torque sensing means being positioned and configured to measure kinesthetic and/or tactile forces/torques acting on the interchangeable tool that interacts with tissue, as well as forces/torques acting on the shaft assembly that interacts with tissue;

d) first position sensing means for tracking position and orientation in 6 degrees of freedom of a tool tip of the interchangeable tool and the intermediate joints of a redundant instrument shaft;

e) second position sensing means for tracking an open/close angle of the interchangeable tool tip;

f) a means of sealing the sensing elements so as to allow the instrument to be sterilized;

g) a computer system incorporating appropriate hardware, software, and algorithms, connected to said force/torque sensing means and said first and second position sensing means, said computer system configured to integrate acquired kinesthetic or tactile force/torque and position information when a user is performing medical procedures;

h) said computer system capable of recording the forces/torques applied to the instrument and the tip position for subsequent analysis or integration into other medical and/or training systems; and

i) said computer system configured to provide feedback to a user of the instrument regarding the instrument position and forces/torques being exerted on tissue undergoing manipulation.

A further understanding of the functional and advantageous aspects of the invention can be realized by reference to the following detailed description and drawings.

### BRIEF DESCRIPTION OF THE DRAWINGS

Embodiments of the present invention are described in greater detail with reference to the accompanying drawings in which:

FIG. 1a shows an instrument design with traditional handle and gripper attachment.

FIG. 1b is a longitudinal cross section of the instrument of FIG. 1a.

FIG. 2a shows an instrument design with needle driver handle and tip.

FIG. 2b shows a longitudinal cross section of the instrument of FIG. 2a.

FIG. 3a is a partial cross sectional view showing details of the instrument design showing o-ring location for attachment of outer shaft.

FIG. 3b is a more detailed view of the area indicated by the arrows in FIG. 3a.

FIG. 4 shows examples of interchangeable tips that can be attached to the instrument.

FIG. 5 shows details of the configuration of the instrument tip. The different tips can be screwed on and off depending on the task to be performed. The inner shaft controls the opening and closing of the tip. The base itself is attached to the middle shaft and the outer shaft protects the sensing elements.

FIG. 6 shows examples of interchangeable handles that can be attached to the instrument.

FIG. 7 details of the configuration of the handle in which the scissor handle and the needle driver handle can be screwed on and off depending on the task to be performed.

FIG. 8 shows an alternative quick-connect configuration of the handle attachment.

FIG. 9 shows the cable wiring to allow for the inner shaft to slide inside the middle shaft in order to accommodate the different tips.

FIG. 10 shows the placement of gauges on the middle shaft.

FIG. 11 shows an alternate configuration of the instrument.

FIG. 12 shows the placement of gauges on the inner shaft.

### DETAILED DESCRIPTION OF THE INVENTION

The systems described herein are directed, in general, to sensorized medical instruments that are suitable for application to, and training and skills assessment for, a variety of therapeutic, diagnostic, surgical and medical procedures.

Although embodiments of the present invention are disclosed herein, the disclosed embodiments are merely exemplary and it should be understood that the invention relates to many alternative forms. Furthermore, the figures are not drawn to scale and some features may be exaggerated or minimized to show details of particular features while related elements may have been eliminated to prevent obscuring novel aspects. Therefore, specific structural and functional details disclosed herein are not to be interpreted as limiting but merely as a basis for the claims and as a representative basis for enabling someone skilled in the art to employ the present invention in a variety of manner. For purposes of instruction and not limitation, the illustrated embodiments are all directed to embodiments of sensorized medical instruments that are suitable for application to, and training and skills assessment for, a variety of therapeutic, diagnostic, surgical and medical procedures.

The present invention provides a system that is suitable for application to, and training and skills assessment for, a variety of therapeutic, diagnostic, surgical and medical procedures. It encompasses a sensorized minimally invasive instrument capable of measuring forces acting at the tip of the instrument as well as the position and orientation of the instrument and the instrument tip. The developed software integrates position and force information to provide feedback to the user.

Thus, the present invention provides a hand-held sensorized minimally invasive instrument. This hand-held instrument has been designed to non-invasively measure the interaction of the instrument with tissue in the form of forces or torques acting in all five degrees of freedom (DOFs) available during MIS. Additional capabilities include the ability to capture instrument tip position and orientation in 6 DOFs. This allows the software to capture the force/torque and position profiles of users performing conventional minimally invasive surgical, therapeutic or diagnostic tasks or during training. The force and position profiles can be used to guide the user of the instrument, ensuring that the instrument is used safely and effectively.

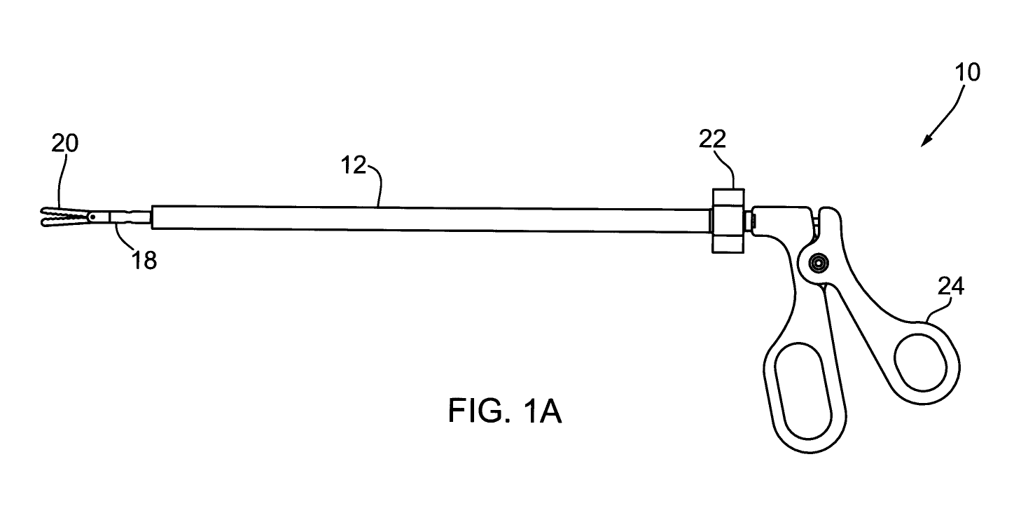

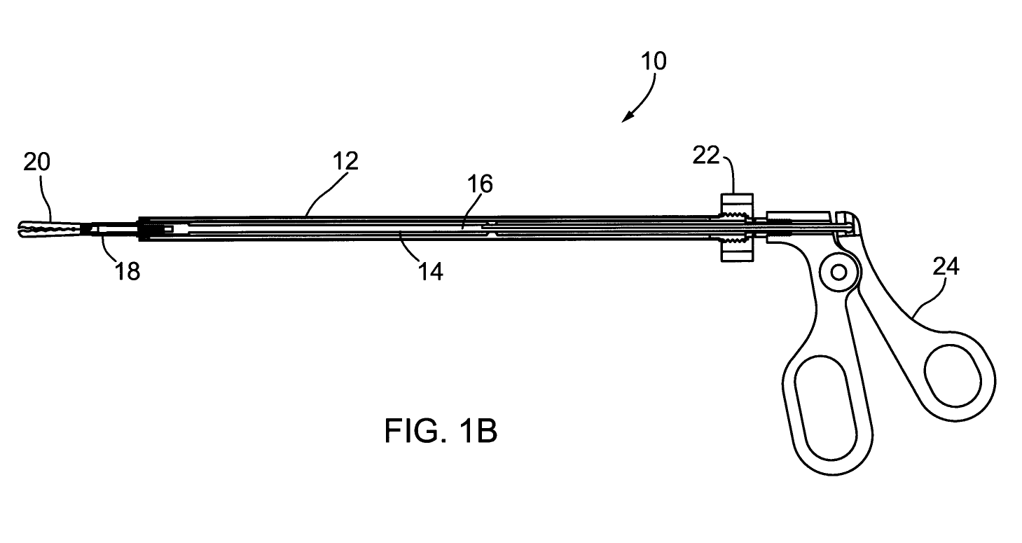

FIGS. 1a, 1b and 2a, 2b show the overall design of the instrument in two different configurations: a typical scissor-like handle and gripper attachment or a needle driver handle and tip. More particularly, FIGS. 1a, 1b shows an instrument design shown generally at 10 with traditional handle 24 and gripper attachment connected to the instrument shafts. The outer shaft 12 protects the sensing elements from physical damage and provides a seal. The distal end of the middle shaft 14 is attached directly to the base of the tip 18. The longitudinal middle shaft contains some of the sensing elements. The proximal end of the middle shaft is connected to a rotating wheel 22 that allows the instrument tip orientation to be adjusted. The distal end of the inner shaft 16 is connected to the moveable components of the instrument tip 20 while the proximal end is coupled to the handle 24. Additional sensing elements are mounted to the longitudinal inner shaft. Position sensing elements may be located on the rotating wheel 22. As the handle 24 is actuated, the inner shaft 16 translates along the longitudinal axis of the instrument relative to the middle shaft 14 and housing 12, causing the instrument tip 20 to open and close, mirroring the motion of the handle 24.

FIGS. 2a and 2b show the same instrument with a needle driver handle 36 and needle driver tip 32/34 connected 30. Similarly, the outer shaft 12 protects the sensing elements from physical damage and provides a seal. The distal end of the middle shaft 14 is attached directly to the base of the tip 32. The proximal end of the middle shaft is connected to a rotating wheel 22 that allows the instrument tip orientation to be adjusted. The distal end of the inner shaft 16 is connected to the moveable components of the instrument tip 34 while the proximal end is coupled to the handle 36. As the handle 36 is actuated, the inner shaft 16 translates along the longitudinal axis of the instrument relative to the middle shaft 14 and housing 12, causing the instrument tip 34 to open and close, mirroring the motion of the handle 36.

In both configurations 10 and 30, the overall appearance, weight and functionality are very similar to traditional hand-held minimally invasive instruments, which is critical for its acceptance among users familiar with non-sensorized instruments and for its seamless integration into current practice. If the instrument is restricted at the entry point, or heavy cables are pulling down on the handle, the normal movement of the instrument will be compromised.

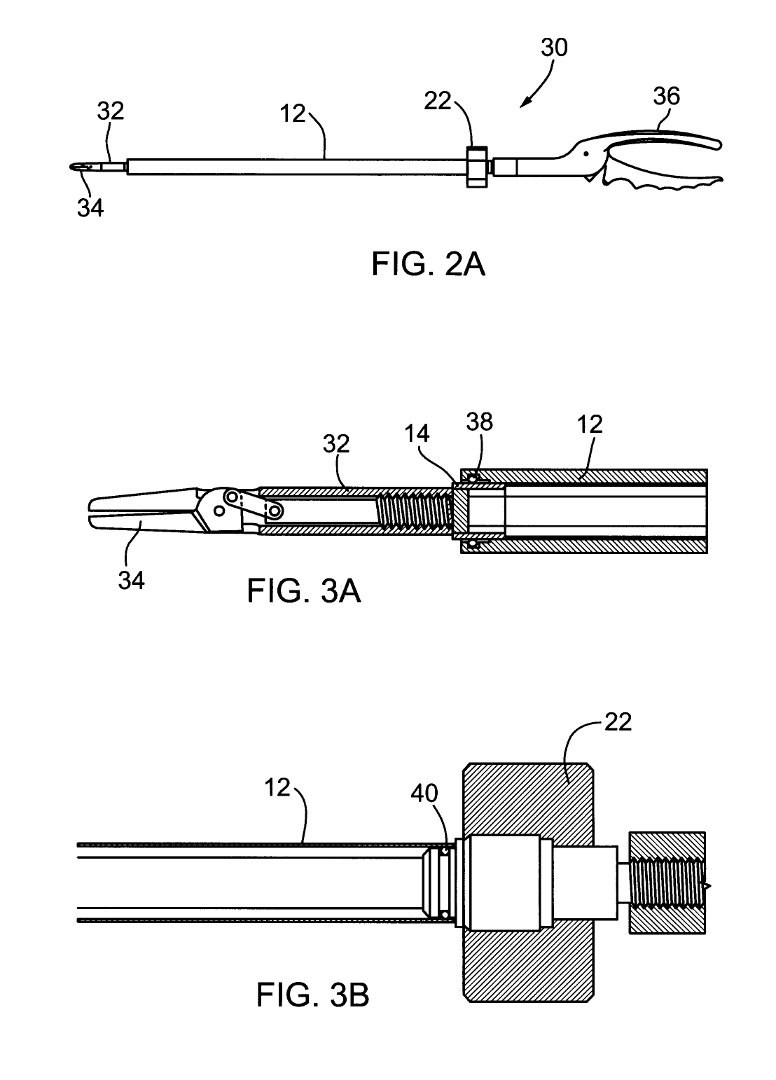

The instrument is comprised of three concentric shafts 12, 14, and 16. An inner shaft 16 controls the opening and closing of the tip 20 or 34 and is directly connected to the handle 24 or 36. A middle shaft 14 provides rigidity to the instrument, connecting the handle 24 or 36 and the tip 18 or 32. An outer shaft 12 “floats” over the middle shaft 14 providing a sealed environment for the sensing elements and protecting them from moisture and wear. The outer shaft 12 is held by two o-rings 38 and 40 shown in FIGS. 3a and 3b which shows the o-ring location for o-ring 40 for attachment of the outer shaft 12 to the handle 24 or 36, (FIG. 3b) at its proximal end, and the o-ring 38 for attachment of the outer shaft 12 to the middle shaft 14 at its distal end (FIG. 3a). The rotating wheel 22 shown in FIG. 3b allows the user to rotate the distal end of the instrument in order to reorient the tip with respect to the handle 24 (FIG. 1), 36 (FIG. 2).

These o-rings 38 and 40 seal the inside of the instrument from moisture and ensure that the outer shaft is firmly held in place. For ease of use, the rotating wheel 22 allows the user to reorient the tip with respect to the handle to optimize ergonomic conditions.

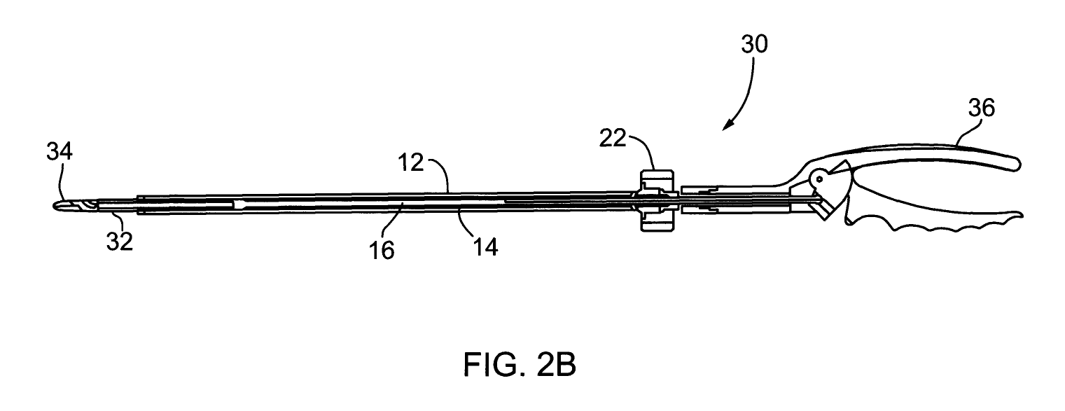

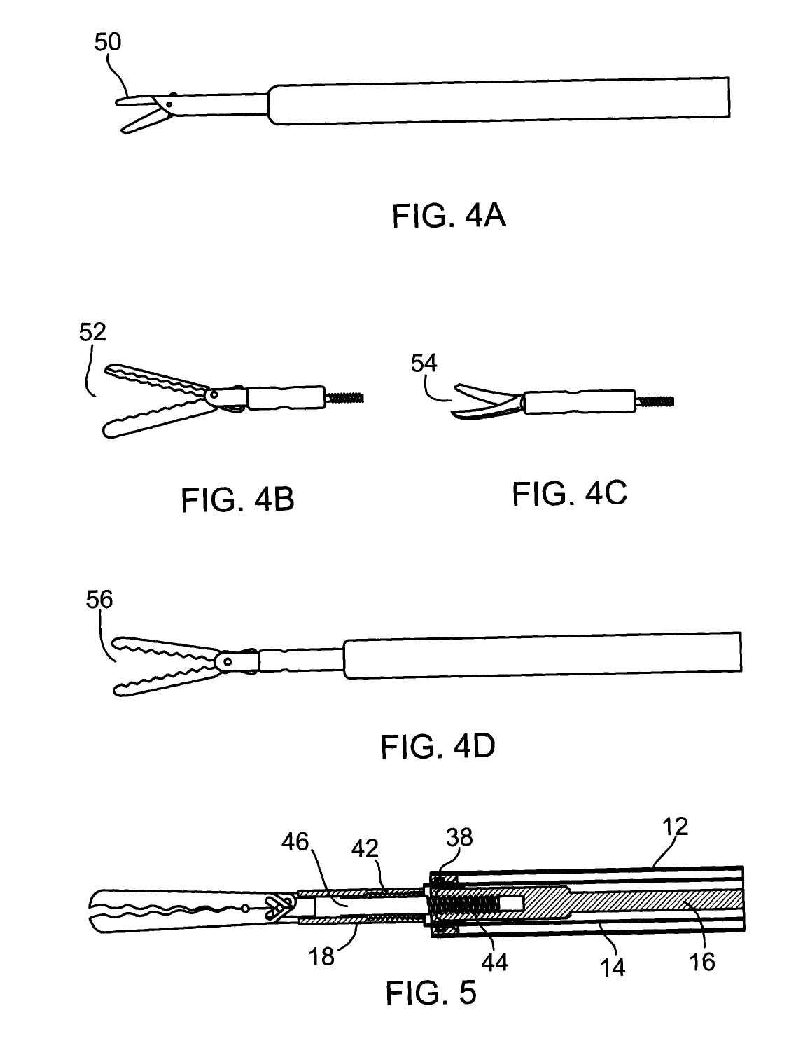

For commercialization purposes, the invention has been designed in a cost-effective and versatile manner with the addition of interchangeable tips and handles. The sensors are all attached to the middle and inner sections of the instrument. This way, the same sensorized elements can be used to perform the wide variety of tasks done during endoscopic surgical procedures by attaching different tips and handles. FIG. 4 shows examples of different interchangeable tips 50, 52, 54 and 56 that can be attached to the same shaft. Details concerning how the tips can be replaced are shown in FIG. 5. This figure presents the fixed assembly components 42 and the moving assembly 44. The fixed assembly 42 comprises the base of the tip 18 which is screwed onto the middle shaft 14 and does not move when he instrument tip is actuated. The moving assembly 44 comprises the tip rod 46 which screws into the inner shaft 16. As the inner shaft 16 is translated by the motion of the handle, the tip is opened and closed accordingly. When the tip is attached to or removed from the instrument, both sections screw on or off at the same time. A variety of different instruments tips, e.g., 50, 52, 54, or 56, can be attached in the same manner, depending on the task to be performed.

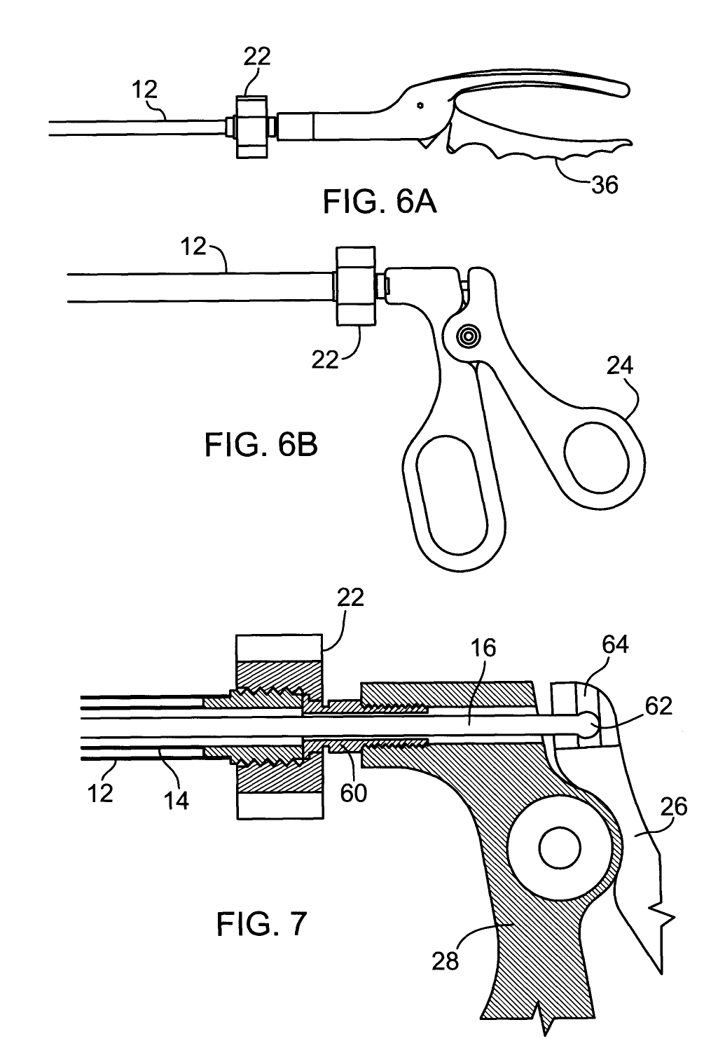

Similarly, the handles of the instruments can also be changed. FIG. 6 shows examples of interchangeable handles 24 and 36 that can be attached to the instrument, in this case with two different handles.

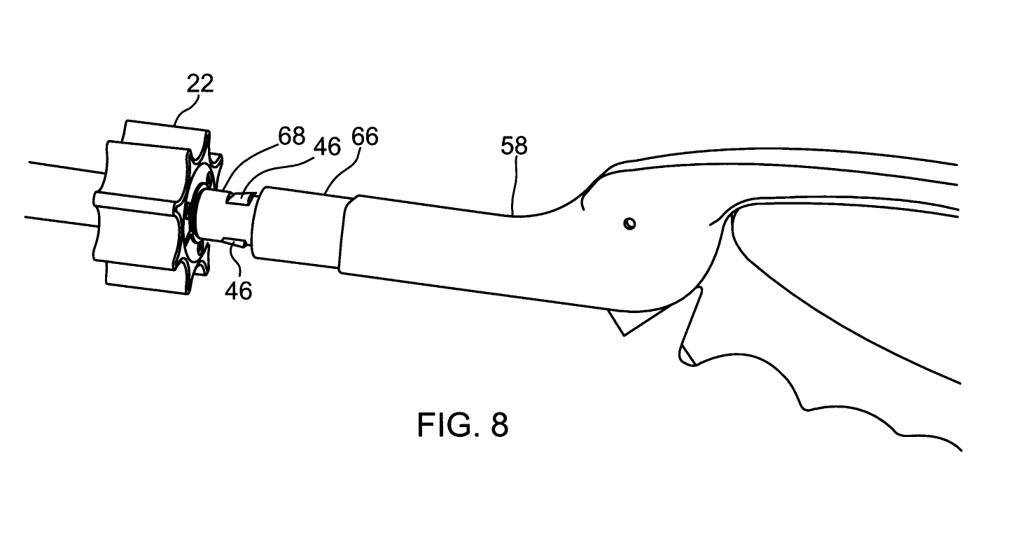

FIG. 7 shows a close-up view of the handle components that allow it to be replaced. The scissor handle and the needle driver handle can be screwed on and off depending on the task to be performed. The figure shows the scissor handle for simplicity, but a variety of different handles could be attached in a similar manner as needed. The inner shaft 16 is coupled to the movable part of the handle 26, by a ball 62 at the proximal end of the inner shaft, which fits into a slot 64 in the movable part of the handle 26. The fixed part of the handle 28 is rigidly attached to a coupler 60. The rotating wheel 22 (attached to the middle shaft 14) rotates with respect to the coupler 60 and allows the distal end of the instrument to be reoriented. By unscrewing the coupler and the rigid part of the handle, the handle assembly can be removed and replaced with a different one.

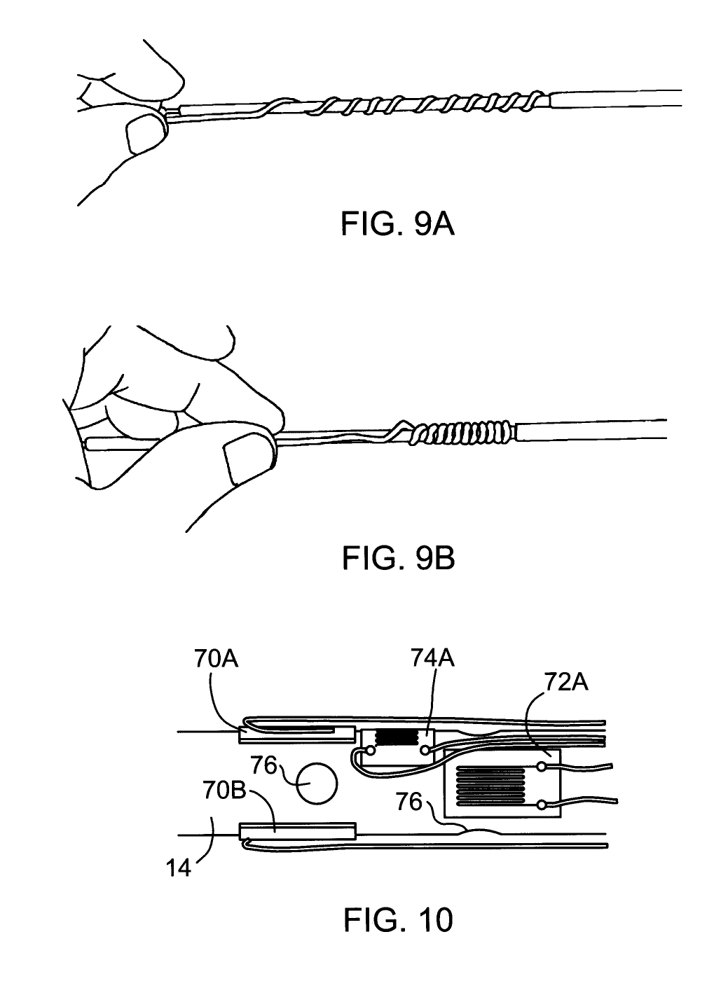

An alternative configuration of the handle attachment is presented in FIG. 8. In this configuration, the fixed part of the handle 58 is rigidly attached to a quick connect 66, which snaps onto the coupler 68. The rotating wheel 22 rotates with respect to the coupler 68. To release the handle, the two tabs 46 are pushed in, which releases the quick connect.

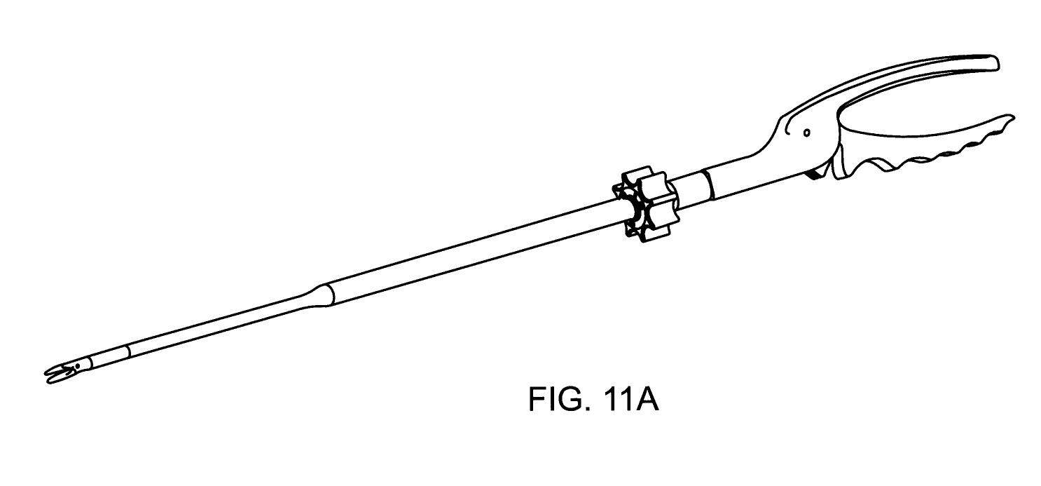

It is recognized that the different handles would require the inner shaft to slide with respect to the middle shaft, while still maintaining its ability to open and close the gripper and without the cables getting tangled. FIG. 9 shows how the cables have been wired to allow the inner shaft to slide inside the middle shaft to allow the instrument tip to be actuated, or if necessary to accommodate different tips and handles. This allows the inner shaft to slide with respect to the middle shaft without causing the cable to get tangled or pinched.

### Sensing Elements

### Force Sensing:

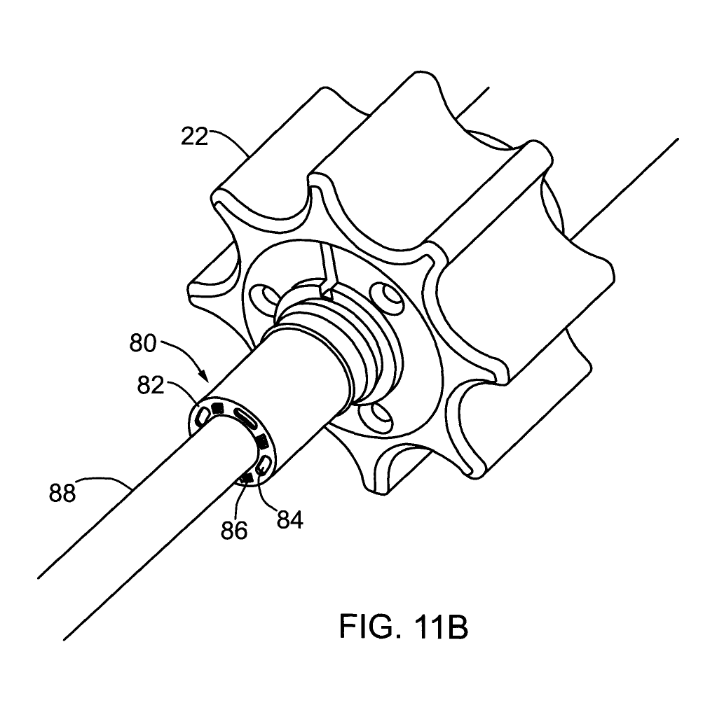

Strain gauges have been incorporated into the middle shaft 14 and the inner shaft 16 of the instrument in order to measure forces in 5 DOFs. FIG. 10 shows a diagram of the surgical tool with the gauges placed on the middle shaft. Two gauges 70A and 70B placed on opposite sides of the shaft are set up in a half-bridge configuration in order to measure the deformation of the shaft in the x direction. Similarly, two other gauges 72A and 72B provide measurement of forces acting in the y direction. Two double rosettes 74A and 74B (one pad that contains two gauges placed at 90 degrees with respect to each other at a 45 degree angle from the center axis of the gauge) are also positioned in the middle in order to measure forces in the axial direction (z) and to measure torsion. These are individually connected in a quarter bridge configuration allowing both axial force and torsion to be resolved, depending on how the signals are combined. In order to properly measure axial forces, four 2.5 mm holes 76 have been drilled to concentrate the deformation in the middle section. The strain gauges 70 and 72 are positioned such that they are centered in between the holes 76. The center of the rosette gauges 74 is located at the middle of the spline that bisects the hole centers.

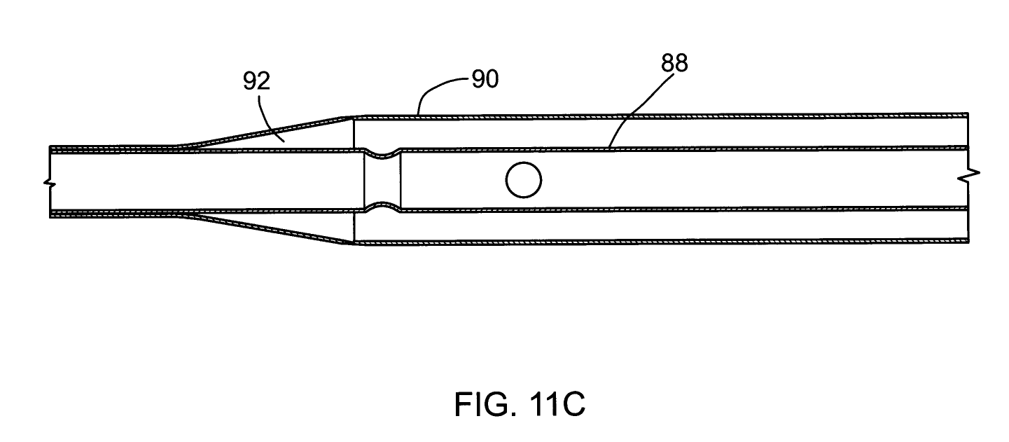

An alternative arrangement for the gauges on the middle shaft, suitable for measuring axial forces, is shown in FIG. 11A. The modifications that characterize this arrangement are detailed in FIGS. 11B and 11C. FIG. 11B shows an additional element 80 that has been added to isolate the axial forces applied to the tip of the instrument. The middle shaft 88 is designed such that the diameter at the proximal end of the shaft is expanded and a flat circular region 82 perpendicular to the central axis of the shaft is produced. Within this flat region, 4 holes 84 equally distributed in a radial pattern are drilled to allow the material to deform when forces are applied to the tip. In the spaces between the holes, four gauges 86 are mounted in a full bridge configuration to measure the deformation of the flat region. The full bridge allows the bending moments and torsion to be cancelled, isolating and amplifying the axial force. The remaining strain gauges on the middle shaft 88 are mounted the same as described for the middle shaft 14, FIG. 10, with the exception that the rosettes 74 are connected in a full bridge configuration to isolate and amplify torsion.

FIG. 11C details an alternative configuration of the outer shaft 90 with a smooth reduction 92 in the shaft diameter in order to prevent any materials from catching on the instrument and to improve visibility and functionality. This transition must be located distally to the placement of the gauges on the middle shaft, in order to provide sufficient space for the placement of the gauges and the routing of the wires to and from the gauges.

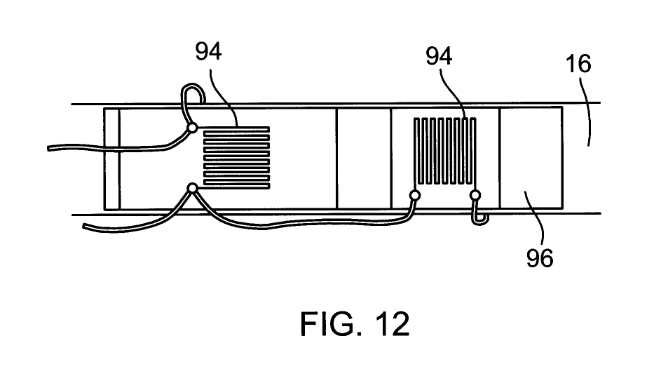

In order to measure gripping and cutting forces, four gauges 94 have been placed on a flattened area 96 of the inner shaft 16 in a full bridge configuration, as shown in FIG. 12. The full bridge configuration allows the bending moment and torsional forces to be cancelled out, isolating and amplifying the axial force. The axial force acting on the inner shaft can be directly related to the gripping or cutting forces acting at the tip.

This method of attaching strain gauges ensures that the forces measured are those acting on the tip of the instrument (interacting with tissue), and not at the hand of the surgeon or at the port location (where the instrument enters the patient's body). It has been shown that minimally invasive instruments do not effectively translate tip forces into the handle forces, making it difficult for the user to feel tissue elasticity [33]. This also means that the forces acting on the handle of the instrument are not an accurate representation of the forces acting on the tissue (which are really the critical forces when it comes to assessing the interaction of the instrument with tissue), and so, the forces acting on the tissue should be measured.

Thus the force/torque sensing means incorporated into the shaft assembly are positioned to provide measurement of kinesthetic or tactile forces in, but not limited to, 5 degrees of freedom (DOFs), with the force/torque sensing means being positioned and configured to measure kinesthetic and/or tactile forces acting on the interchangeable surgical tool that interacts with tissue.

### Position Sensing:

A means of providing position feedback is required to ensure that the position of the instrument tip can always be relayed to the user, even if the instrument is not directly visible or within the field of view of an endoscopic camera. This can be accomplished through the use of commercially available tracking systems. The objective of any tracking system is to provide the position and orientation (pose) of an object. This usually involves attaching a sensor to the object of interest and using specialized hardware and software algorithms to determine the pose.

The most popular tracking systems used in modern interventions are optical and electromagnetic. Optical tracking systems (OTS) are very accurate but require an unobstructed line-of-sight between the surgical instruments and the sensor, which is not feasible inside the patient's body. Electromagnetic tracking systems (EMTS) do not require an unobstructed line-of-sight and thus allow for unrestricted handling within their working volume. The sensors can be easily attached to any instrument and their poses tracked inside and outside of the patient. An electromagnetic transmitter situated outside of the body generates a weak spatially-varying magnetic field that can be measured by the sensor to dynamically compute position and orientation. The greatest drawback with using electromagnetic trackers is that they can suffer from magnetic field distortions caused by the presence of conductive metals within their working volume.

Although these tracking systems are easy to integrate and provide good tracking accuracy, a significant limitation in their use is that they are very expensive and would hinder the commercialization of the proposed invention. An alternative for tracking position involves the use of software that processes the real-time images obtained from the endoscope. A detailed description of existing progress in this area is included in the Section entitled “Prior Art.” We have done some work to incorporate image processing to track the position of the instrument tip which has been successfully integrated into the system for 2 dimensional tracking.

The invention also involves software that converts the signals into a proper output for user guidance. It could involve a real-time interface that incorporates image guidance with the force and position information from the instrument in order to guide the user to specific areas within the body or to prevent the application of excessive forces in critical areas. For training purposes, the force/torque and position profiles captured by the instrumented tool during a procedure decomposed into tasks and sub-tasks can be used to statistically model and analyze the learning curves and skill levels of residents and surgeons during MIS. The inventors also contemplate this system to be of value in enabling a novice surgeon to gain an objective kinesthetic understanding of force and position profiles that are desired for certain tasks based on analyzing the profiles of experienced surgeons. In fact, objective skills assessment and learning curve modeling provide a means for constructive feedback to a trainee about various performance aspects such as the appropriate application of forces and torques and desired spatial trajectories.

Since the instrument is suitable for use in all environments (e.g., training box, animals or humans), it may be used to collect data suitable for the quantification of various procedures and the skill level of the practitioner or trainee. This information may be used to develop surgical-assist and training environments that utilize virtual fixtures to support the user as required to enhance outcomes and accelerate training.

### Additional Embodiments

Other modifications to the present invention include the following. Although the current prototype measures forces in 5 DOFs, the same technology can easily be applied to measure forces in six or seven DOFs acting at the tip (the seventh DOF being the gripping or cutting force), and six or seven DOFs acting at the handle. Similarly, the same position sensing technology can be used to measure the position of the instrument tip in five, six or seven DOFs (the seventh DOF being the opening and closing of the tip).

The invention can be extended to instruments with dexterous wrists, where a full 6 degrees of mobility are available inside the patient's body. In that case, sensing of forces and torques acting on all 6 DOFs is required, plus gripping forces.

The invention may be further extended to instruments that provide redundant degrees of freedom (7, 8 or more DOFs) within the patient's body, allowing the instrument to manoeuvre through and/or around complex anatomy. In this case, to measure tool-tissue interaction forces, sensors must be placed at the tip or the last link of the instrument. Additional sensors could be included within intermediate links if the forces applied to non-target anatomy (i.e., anatomy being manoeuvred around or through) are also of interest.

Instead of sensing forces on the inner shaft to measure kinesthetic forces acting at the gripper, it is possible to incorporate tactile sensors into the individual grippers.

The opening and closing of the instrument tip may be measured using an internal sensor, located within the instrument shaft. For example, the position sensing means for the open/close angle of the interchangeable instrument tip may be a linear potentiometer, linear variable differential transformer, contact switches, optical encoders, or image-based/optical flow type systems to mention a few. The motion of the inner shaft can be tracked directly using, for example, the linear potentiometer or linear variable differential transformer (LVDT). By placing markings or etchings on the surface of the inner shaft, an optical encoder-type system could be employed. Similarly (although with much lower resolution), the position of a bump (or groove) on the inner shaft could be detected using a series of contact switches.

Another approach would track the surface texture of the inner shaft using a small image sensor (camera) and light source (LED) in combination with optical flow algorithms, similar to the operation of an optical mouse. In each case, the sensors would be mounted on the middle shaft (on the inner surface or through a hole).

In place of standard access to the surgical, therapeutic or diagnostic site (incision in a patient with a trocar inserted into it or hole in a training box), a sensorized trocar could be employed. This would allow the forces developed at the port to be measured. Measurement approaches include the use of a pressure-sensitive liner (a strip containing an array of pressure-sensitive elements that runs along the circumference of the trocar opening) or strain gauges mounted on flexible members that connect inner and outer concentric rings. In the latter case, the inner ring would move with the instrument, while the outer ring would remain fixed to the port.

The trocar may be rigidly attached to a training box, eliminating trocar-hole interaction forces. In a clinical setting, the trocar could be partially constrained by suturing the outer ring to the surrounding tissue. Additionally, the trocar-incision interaction forces could be measured by mounting a pressure-sensitive strip on the outside circumference of the trocar.

The sensorized trocar could be used to track the instrument position and orientation. Optical sensors could read markings on the instrument shaft to measure the roll (about the z-axis) and instrument depth (z-axis). Alternatively, an optical flow technique, using a small image sensor and light source, could track surface texture of the outer shaft. In place an optical approach, sensors that rely on mechanical contact could be used. Additionally, the instrument could be incorporated into a system that uses rollers or balls in contact with the instrument shaft to transfer motion to rotary encoders or potentiometers (e.g., [18]). The pitch (about the x-axis) and yaw (about the y-axis) of the instrument may be measured using a “floating” inner ring or “fingers” that move with the instrument.

If necessary, the movement of the trocar in the incision may be measured internally using accelerometers or gyroscopes or externally using an EM tracker or optical tracking system.

The same technology can be applied to instruments that are held by robotic systems in a master-slave configuration, in order to provide haptic feedback to the user during robot-assisted minimally invasive procedures. In this configuration, the sensorized instrument would form part of the slave (with the surgical robot providing actuation) and force feedback mechanisms would be added at the master side.

The same technology can be incorporated into traditional handheld surgical instruments (not endoscopic instruments) in order to provide feedback during open surgery procedures.

The instruments described above (hand-held, robotically-held or master-slave) could be used for measurements in real surgical procedures.

The sensorized instrument described above is suitable for conventional minimally invasive surgery, therapy and diagnosis. The sensorized instruments can provide real-time information regarding tip-tissue interaction forces that, in combination with position data, can be superimposed on images in the video monitors showing the output of one or more endoscopes.

The strain gauges can be sealed in a biocompatible film to protect against the infiltration of water and/or cleaning fluids. Furthermore, the outer shaft serves to provide a sealed environment for the gauges, providing an additional degree of protection. These design features, along with the use of biocompatible materials for the manufacture of the instrument tips, shafts and handles ensure that the instrument can be sterilized prior to clinical use.

There are several advantages and unique features of the invention that can be summarized as follows. The system measures all of the forces and torques acting in all five (5) degrees of freedom available during minimally invasive surgery. This is of particular importance during a suturing task, since due to its complexity and the required precision when handling needles and delicate tissues, force measurement in all degrees of freedom would be very useful.

The system is also capable of providing position feedback, in as many degrees of freedom (up to six) as required for the task.

In spite of containing force sensing elements, the instrument is similar in shape, size and weight to traditional minimally invasive instruments. A small, lightweight and flexible cable is attached to the shaft so that no limiting forces are acting on the shaft that could affect the way the instrument is moved.

The forces being measured are those acting on the tip of the instrument and not on the handle or at the port location. The forces acting at the tip are those that are directly being transferred to the tissue, and so are the critical forces that need to be measured to produce accurate measurements.

Replaceable tips and handles make the instruments more affordable since all of the sensing elements are on the shaft and multiple tasks can be performed by the addition of lower cost tips and handles.

The sensing elements incorporated into the design are sealed to allow the instrument to be sterilized according to accepted practices. This is a critical requirement for its use in a clinical setting (i.e., for use in humans).

The rest of the system is a software package that can be installed on any computer, reducing the need for other expensive equipment that could limit its commercialization capabilities.

Considering the above advantages and unique features, the system disclosed herein is believed to be the only one of its kind for self-contained monitoring and recording of tool-tissue interaction during clinical procedures. Its compact, light-weight design, combined with a highly versatile and cost-effective configuration, ensure the effectiveness and commercialization potential of the system.

As used herein, the terms “comprises”, “comprising”, “including” and “includes” are to be construed as being inclusive and open ended, and not exclusive. Specifically, when used in this specification including claims, the terms “comprises”, “comprising”, “including” and “includes” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

### REFERENCES

[1] S. Shimachi, Y. Fujiwara, and Y. Hakozaki, “New sensing method of force acting on instrument for laparoscopic robot surgery,”International Conference on Computer Assisted Radiology and Surgery, International Congress Series vol. 1268, 2004, pp. 775-780.
[2] M. Tavakoli, R. V. Patel, and M. Moallem, “Haptic interaction in robot-assisted endoscopic surgery: a sensorized end-effector,” International Journal of Medical Robotics and Computer Assisted Surgery, vol. 1, no. 2, 2005, pp. 53-63.
[3] J. Dargahi, S. Payandeh, and M. Parameswaran, “A micromachined piezoelectric teeth-like laparoscopic tactile sensor: theory, fabrication and experiments,” Proceedings of the 1999 IEEE International Conference on Robotics and Automation, Detroit, Mich., May 1999, pp. 299-304.
[4] K. Takashima, K. Yoshinaka, T. Okazaki, and K. Ikeuchi, “An endoscopic tactile sensor for low invasive surgery,” Sensors and Actuators A, vol. 119, 2005, pp. 372-383.
[5] F. Van Meer, D. Esteve, A. Giraud, and A. M. Gué, “Si-micromachined 2D force sensor for a laparoscopic instrument,” International Conference on Computer Assisted Radiology and Surgery, International Congress Series vol. 1268, 2004, pp. 1334.
[6] G Tholey, A. Pillarisetti, W. Green, and J. P. Desai, “Design, development, and testing of an automated laparoscopic grasper with 3-D force measurement capability,” in S. Cotin and M Metaxas (eds), ISMA 2004, LNCS 3078, pp. 38-48, 2004.
[7] H. Singh, R. Sedaghati, and J. Dargahi, “Experimental and finite element analysis of an endoscopic tooth-like tactile sensor,” Sensors, vol. 1, pp. 259-264, 2003.
[8] P. Dubois, “In vivo measurement of surgical gestures,” IEEE Transactions on Biomedical Engineering, vol. 49, no. 1, January 2002, pp. 49-54.
[9] C. Basdogan, S. De, and J. Kim, “Haptics in minimally invasive surgical simulation and training,” IEEE Transactions on Computer Graphics and Applications, March/April 2004. Search for proper reference.
[10] J. Rosen, and B. Hannaford, “Markov modeling of minimally invasive surgery based on tool/tissue interaction and force/torque signatures for evaluating surgical skills,” IEEE transactions on Biomedical Engineering, vol. 48, no. 5, May 2001, pp. 579-591.
[11] Bicchi, G. Canepa, D. De Rossi, P. Iacconi, and E. P. Scilingo, “A Sensorized minimally invasive surgery tool for detecting tissutal elastic properties,” Proceedings of the 1996 IEEE International Conference on Robotics and Automation, Minneapolis, Minn., April 1996.
[12] P. J. Berkelman, L. L. Whitcomb. R. H. taylor, and P. Jensen, “A miniature microsurgical instrument tip force sensor for enhanced force feedback during robot-assisted manipulation,” IEEE Transactions on Robotics and Automation, vol. 19, no. 5, pp. 917-922, October, 2003.
[13] Hannaford, J. Trujillo, M. Sinanan, M. Moreyra, J. Rosen, J. Brown, R. Leuschke, and M. MacFarlane, “Computerized endoscopic surgical grasper,” Proceedings of Medicine Meets Virtual Reality, San Diego, Calif., January 1998, pp 111-117.
[14] J. Rosen, and B. Hannaford, “Force controlled and teleoperated endoscopic grasper for minimally invasive surgery—experimental performance evaluation,” IEEE Transactions on Biomedical Engineering, vol. 46, no. 10, October 1999, pp. 1212-1221.
[15] H. Mayer, I. Nagy, A. Knoll, E. Schirmbeck, and R. Bauernschmitt, “The EndoPAR system for minimally invasive robotic surgery,” Proceedings of the IEEE/RSJ International Conference on Intelligent Robots and Systems, Sendal japan, Sep. 28-Oct. 2, 2004, pp. 3637-3642.
[16] J. Rosen, J. D. Brown, L. Chang, M. Barreca, M. Sinanan, and B. Hannaford, “The BlueDRAGON—a system for measuring the kinematics and the dynamics of minimally invasive surgical tools in-vivo,” Proceedings of the IEEE International Conference on Robotics and Automation, Washington, D.C., May 2002, pp. 1876-1881.
[17] J. Rosen, M. Solazzo, B. Hannaford, and M. Sinanan, “Task decomposition of laparoscopic surgery for objective evaluation of surgical residents' learning curve using hidden Markov model,” Computer Aided Surgery, vol. 7, 2002, pp. 49-61.
[18] B. Hannaford, J. Rosen, J. D. Brown, T. M. Kowalewski, M. Sinanan, and L. Chang, “Skill Evaluation,” Patent Application Publication No. U.S. 2007/0172803 A1, July 26, 2007.
[19] Wellman, “Tactile Imaging,” PhD Thesis, Harvard University, 2001.
[20] P. E. Dupont, R. D. Howe, P. J. del Nido, P. M. Novotny, J. A. Stoll, and N. V. Vasilyev, “GPU based real-time instrument tracking with three-dimensional ultrasound,” International Conference on Medical Image Computing and Computer Assisted Intervention (MICCAI'06), vol. 9, part 1, pp. 58-65,2006.
[21] D. R. Uecker, Y. Wang, and Y. F. Wang, “A new framework for vision enabled and robotically assisted minimally invasive surgery,” Computerized Medical Imaging and Graphics, vol. 22, no. 6, pp. 429-37, 1998.
[22] M. Ribo, “State of the Art Report on Optical Tracking,” Tech. Rep, Vienna, Austria, 2001.
[23] A. Frederick, M. P. Fried, H. Gopal, B. T. Ho, J. Kleefield, Kuhn, and E. Reardon, “Image-guided endoscopic surgery: results of accuracy and performance in a multicenter clinical study using an electromagnetic tracking system,” The Laryngoscope, vol. 107, no. 5, pp 594-601, May 1997.
[24] D. Burschka, J. J. Corso, L. Dewan, G. D. Hager, C. Hasser, B. Hoffman, D. Larkin, W. Lau, M. Li, H. Lin, P. Marayong, and N. Ramey, “Navigating inner space: 3-D assistance for minimally invasive surgery, robotics and autonomous systems,” Advances in Robot Vision, vol. 52, no. 1, pp. 5-26, July 2005.
[25] L. Adams, M. Von Düring, B. Fricke, A. G. Harders, A. Hentsch, L. Heuser, W, Konen, S. Tombrock, and M. Scholz, “Development of an endoscopic navigation system based on digital image processing,” Computer Aided Surgery, vol. 3, no. 3, pp. 134-143, 1998.
[26] C. Lee, D. R. Uecker, Y. Wang, and Y. F. Wang, “Image analysis for automated tracking in robot-assisted endoscopic surgery,” Pattern Recognition, vol. 1—Conference A: Proceedings of the 12th IAPR International Conference on Computer Vision & Image Processing, vol. 1, pp. 88-92, Oct. 9-13, 1994.
[27] A. Chung, F. Deligianni, and G. Z. Yang, “Pq-space based 2D/3D registration for Endoscope tracking,” International Conference on Medical Image Computing and Computer Assisted Intervention (MICCAI'03), vol. 1, pp. 311-318, October 2003.
[28] Z. Hammoud, S. Miller, W. Peine, and J. Son, “Tactile imaging system for localizing lung nodules during video assisted thoroscopic surgery,” 2007 IEEE International Conference on Robotics and Automation, Roma, Italy, April 10-14, 2007.
[29] C. Doignon, J. Gangloff, J. Leroy, A. Krupa, J. Marescaux, M. F. de Mathelin, G. Morel, and L. Soler, “Autonomous 3-D positioning of surgical instruments in robotized laparoscopic surgery using visual servoing,” IEEE Transactions on Robotics and Automation, vol. 19, no. 5, pp. 842-853, October 2003.
[30] K. Arbter, G. Hirzinger, and G. Q. Wei, “Real-time visual servoing for laparoscopic surgery. Controlling robot motion with color image segmentation,” IEEE Engineering in Medicine and Biology Magazine, vol. 16, no. 1, pp 40-45, January 1997.
[31] P. Cinquin, J.-A. Long, E. Orvain and S. Voros, “Automatic detection of instruments in laparoscopic images: a first step towards high level command of robotized endoscopic holders,” The First IEEE/RAS-EMBS International Conference on Biomedical Robotics and Biomechatronics (BioRob 2006), pp. 1107-1112, February 20-22, 2006.
[32] P. Dario, G. Megali, R. U. Thoranaghatte, and O. Tonet, “Tracking endoscopic instruments without a localizer: A shape analysis based approach,” Computer Aided Surgery, vol. 12, no. 1, pp. 35-42, 2007.
[33] V. Gupta, N. P. Reddy, and P. Batur, “Forces in surgical tools: comparison between laparoscopic and surgical forceps,” Proceedings of the 18^{th} Annual Conference of the IEEE Engineering in Medicine and Biology Society, Amsterdam, 1996, pp. 223-224.

1. A sensorized instrument system, comprising:
a) an instrument with an interchangeable handle, a shaft assembly operably connected at a proximal end portion to the interchangeable handle and attached at a distal end portion to an interchangeable tool, said interchangeable handle, shaft assembly and interchangeable tool operably connected so that the interchangeable handle controls actions of the interchangeable tool, said shaft assembly being configured to rotate the tool with respect to the interchangeable handle;
b) said instrument capable of accommodating tools for surgical, diagnostic, therapeutic and general medical procedures;
c) force/torque sensing means incorporated into the shaft assembly and positioned to provide measurement of kinesthetic or tactile forces/torques in, but not limited to, 5 degrees of freedom (DOFs), said force/torque sensing means being positioned and configured to measure kinesthetic and/or tactile forces/torques acting on the interchangeable tool that interacts with tissue;
d) first position sensing means for tracking position and orientation in 6 degrees of freedom (DOFs) of a tool tip of the interchangeable tool;
e) second position sensing means for tracking an open/close angle of the interchangeable tool tip;
f) a means of sealing the sensing elements so as to allow the instrument to be sterilized;
g) a computer system incorporating appropriate hardware, software, and algorithms,connected to said sensing means and said first and second position sensing means, said computer system configured to integrate acquired kinesthetic or tactile force/torque and position information when a user is performing medical procedures;
h) said computer system configured to record forces/torques applied to the instrument and a tool tip position for subsequent analysis and/or integration into other medical and/or training systems; and
i) said computer system configured to provide feedback to a user of the instrument regarding the instrument position and forces being exerted on tissue undergoing manipulation.

2. The system according to claim 1 wherein said instrument is a handheld medical instrument.

3. The system according to claim 1 wherein said instrument is configured to be held by a surgical robot and wherein said computer system is configured to provide haptic feedback during robot-assisted surgical procedures.

4. The system according to claim 1 wherein said instrument is configured to be held by a surgical robot in a master-slave configuration, and said force/torque sensing means being configured to give haptic feedback to the user.

5. The system according to claim 1 wherein said instrument is configured to be held by a surgical robot in a master-slave robot configuration, and said force/torque sensing means being connected to force feedback mechanisms on the master robot.

6. The system according to claim 1 used in surgical simulation environments for training.

7. The system according to claim 1 used in animal based training environments.

8. The system according to claim 1 used in surgery, therapy, diagnosis or surgical training performed on humans.

9. The system according to claim 1 wherein said computer system is configured to allow surgical tasks to be decomposed in order to quantify similarities or differences between models representing surgical trainees at various stages of training.

10. The system according to claim 1 wherein said force/torque sensing means are strain gauges.

11. The system according to claim 1 wherein said force/torque sensing means are tactile sensors and/or a combination of strain gauges.

12. The system according to claim 1 wherein said first and second position sensing includes any one or combination of accelerometers, gyroscopes, electromagnetic trackers, optical or vision-based tracking systems, configured to track movement of the instrument.

13. The system according to claim 1 wherein said second position sensing means for the open/close angle of the interchangeable instrument tip is a linear potentiometer, linear variable differential transformer, contact switches, optical encoder, image-based or optical-flow type system.

14. The system according to claim 1 including a sensorized trocar comprised of a linear strip containing an array of pressure-sensitive elements that runs along an inner circumference of the trocar opening to measure tool-trocar interaction forces.

15. The system according to claim 1 including a sensorized trocar comprised of strain gauges mounted on flexible members that connect inner and outer concentric rings of the trocar, wherein in use the inner ring moves with the instrument, facilitating measuring tool-trocar interaction forces.

16. The system according to claim 14 wherein the trocar is rigidly attached to a training box, eliminating trocar-hole interaction forces.

17. The system according to claim 14 wherein the trocar includes a pressure-sensitive strip on an outside circumference of the trocar for measuring trocar-incision interaction forces.

18. The system according to claim 14 wherein the sensorized trocar is configured to track the position and orientation of the instrument.

19. The system according to claim 1 including markings on a shaft form part of said shaft assembly of the instrument which are optically readable to measure roll (about the z-axis) and instrument depth (z-axis).

20. The system according to claim 1 including texture features on a shaft of said shaft assembly of the instrument which are optically readable to measure roll (about the z-axis) and instrument depth (along the z-axis), or can be detected by mechanical contact.

21. A sensorized instrument system, comprising:
a) a hand-held minimally invasive instrument with an interchangeable handle, an inner shaft attached at a proximal end to the interchangeable handle and attached at a distal end to an interchangeable tool, said interchangeable handle, inner shaft and interchangeable minimally invasive tool operably connected so that the interchangeable handle controls actions of the interchangeable tool;
b) said minimally invasive instrument capable of accommodating tools for surgical, diagnostic, therapeutic and general medical procedures;
c) a middle shaft in which the inner shaft is movable for connecting the interchangeable handle and the interchangeable minimally invasive tool;
d) at least one force/torque sensing means incorporated into the middle and the inner shafts and positioned to provide measurement of kinesthetic or tactile forces/torques in at least 5 degrees of freedom (DOFs), said plurality of at least one force/torque sensing means being positioned and configured to measure kinesthetic or tactile forces/torques acting on the interchangeable minimally invasive tool that interacts with tissue;
e) first position sensing means for tracking position and orientation in 6 degrees of freedom of a tool tip of the interchangeable minimally invasive tool and the intermediate joints of a redundant surgical shaft;
f) second position sensing means for tracking an open/close angle of the instrument tip;
g) a means of sealing the sensing elements so as to allow the minimally invasive instrument to be sterilized;
h) a computer system incorporating appropriate hardware, software, and algorithms, connected to said force/torque sensing means and said first and second position sensing means, said computer system configured to integrate acquired kinesthetic or tactile force/torque and position information when a user is performing medical procedures; and
i) said computer system capable of recording the forces/torques applied to the instrument and the tip position for subsequent analysis or integration into other medical and/or training systems; and
j) said computer system configured to provide feedback to a user of the instrument regarding the instrument position and forces/torques being exerted on tissue undergoing manipulation.

22-35. (canceled)

36. The system according to claim 15 wherein the trocar is rigidly attached to a training box, eliminating trocar-hole interaction forces.

37. The system according to claim 15 wherein the trocar includes a pressure-sensitive strip on an outside circumference of the trocar for measuring trocar-incision interaction forces.

38. The system according to claim 15 wherein the sensorized trocar is configured to track the position and orientation of the instrument.

39. (canceled)

40. (canceled)

41. A sensorized instrument system, comprising:
a) an instrument that articulates to provide additional degrees of degrees of freedom beyond 5 degrees of freedom of motion available with a conventional minimally invasive instrument, including an interchangeable handle, a shaft assembly operably connected at a proximal end portion to the interchangeable handle and attached at a distal end portion to an interchangeable tool, said interchangeable handle, shaft assembly and interchangeable tool operably connected so that the interchangeable handle controls actions of the interchangeable tool, said shaft assembly being configured to rotate the interchangeable tool with respect to the interchangeable handle;
b) said instrument capable of accommodating tools for surgical, diagnostic, therapeutic and general medical procedures;
c) force/torque sensing means incorporated into the shaft assembly and positioned to provide measurement of kinesthetic or tactile forces/torques in any number of degrees of freedom, said force/torque sensing means being positioned and configured to measure kinesthetic and/or tactile forces/torques acting on the interchangeable tool that interacts with tissue, as well as forces/torques acting on the shaft assembly that interacts with tissue;
d) first position sensing means for tracking position and orientation in 6 degrees of freedom of a tool tip of the interchangeable tool and the intermediate joints of a redundant instrument shaft;
e) second position sensing means for tracking an open/close angle of the interchangeable tool tip;
f) a means of sealing the sensing elements so as to allow the instrument to be sterilized;
g) a computer system incorporating appropriate hardware, software, and algorithms, connected to said force/torque sensing means and said first and second position sensing means, said computer system configured to integrate acquired kinesthetic or tactile force/torque and position information when a user is performing medical procedures;
h) said computer system capable of recording the forces/torques applied to the instrument and the tip position for subsequent analysis or integration into other medical and/or training systems; and
i) said computer system configured to provide feedback to a user of the instrument regarding the instrument position and forces/torques being exerted on tissue undergoing manipulation.

42-60. (canceled)
